(19) United States
(12) Patent Application Publication
(10) Pub. No.: US 2025/0325082 A1
(43) Pub. Date: Oct. 23, 2025

(54) SECURE STORAGE DEVICE WITH BIOMETRIC LOCKING MECHANISM

(71) Applicant: DAT SAGE TECH SOLUTIONS LLC, Shelton, CT (US)

(72) Inventors: Gisele Tyler, Shelton, CT (US); Trevor Dyment, Boston, MA (US); Aidan Rogers, New York, NY (US); Devon Tyler, New York, NY (US)

(73) Assignee: DAT SAGE TECH SOLUTIONS LLC, Shelton, CT (US)

(21) Appl. No.: 19/182,528

(22) Filed: Apr. 17, 2025

Related U.S. Application Data
(60) Provisional application No. 63/635,611, filed on Apr. 17, 2024.

(51) Int. Cl. A45C 13/18; A45C 11/24; A45C 13/02; A45C 13/10; A61B 5/1172; G07C 9/00

(52) U.S. Cl. CPC A45C 13/18; A45C 11/24; A45C 13/02; A45C 13/103; A61B 5/1172; G07C 9/00563; A45C 2013/026

(57) ABSTRACT

The present invention relates to a storage device comprising one or more compartments, each including a securing mechanism disposed along its perimeter. At least one of the compartments may include a divider system comprising one or more slots and dividers configured to define customizable storage sections. The device may further include a locking mechanism comprising a latch and a fingerprint sensor, the locking mechanism being operable to selectively secure the securing mechanism such that access to the compartment is restricted when locked and permitted when unlocked.

### FIELD OF INVENTION

The present disclosure is in the field of storage devices. Specifically, storage devices utilizing securable locking mechanisms.

### INTRODUCTION

Previous approaches to storage devices have typically involved simplistic designs with limited security features. Conventional storage devices often comprise basic compartments that rely solely on mechanical latches, padlocks, or key-based systems for access control, which offer limited protection against unauthorized access or tampering. As a result, such devices are frequently inadequate for storing valuable, sensitive, or hazardous materials, including medications and personal items requiring secure containment.

In addition, while some advanced locking solutions have been proposed, they rarely integrate biometric security features-such as fingerprint sensors-into portable or customizable storage devices. The lack of such features limits the potential for secure, user-specific access control. Accordingly, existing solutions fail to offer a comprehensive system that simultaneously provides robust security, internal customization, and modern access technology.

Some prior storage devices have incorporated only basic divider systems to organize the internal space within compartments. However, these dividers are often fixed, creating static storage sections that limit the flexibility and adaptability of the internal layout. Without adjustable or modular compartmentalization or divider, users are unable to efficiently store objects of varying shapes and sizes, reducing the overall versatility of the device and leading to inefficient use of available space.

There remains a need for a storage device that integrates advanced security mechanisms, including biometric authentication, with modular, user-adaptable compartment designs to offer enhanced protection, flexibility, and usability in a single, cohesive structure.

### BRIEF DESCRIPTION OF THE DRAWINGS

The accompanying drawings, which are incorporated in and constitute a part of this specification, illustrate exemplary embodiments of the present disclosure and, together with the detailed description, serve to explain the principles of the disclosure.

FIG. 1 illustrates a front perspective view of a storage device including a first compartment and a second compartment, according to an embodiment of the present disclosure.

FIG. 2 illustrates a close-up view of a locking mechanism positioned between the upper and lower compartments, showing the biometric authentication module and associated zipper tab interface according to an embodiment of the present disclosure.

FIG. 3 illustrates a top-down view of the first compartment in an open configuration, showing internal divider structures, elastic securing bands, and accessory pouches, according to an embodiment of the present disclosure.

FIG. 4 illustrates a top-down view of the second compartment in an open configuration, showing customizable internal depth and alternative storage configurations according to an embodiment of the present disclosure.

FIG. 5 illustrates a perspective view of the second compartment, showing one or more open-ended pouches on the lid and a customizable divider system on the base, according to an embodiment of the present disclosure.

FIG. 6 illustrates an exploded view of a removable divider system, including interlocking dividers with integrated slot geometries for forming a modular internal storage grid according to an embodiment of the present disclosure.

### DETAILED DESCRIPTION

In the following detailed description, reference is made to the accompanying drawings, in which like numerals designate identical or functionally similar elements. The drawings are provided for purposes of illustration only and should not be construed as limiting the scope of the disclosure. These described implementations are presented in sufficient detail to enable those skilled in the art to practice the invention, it being understood that other implementations may be utilized, and that structural or functional modifications and/or substitutions of various elements may be made without departing from the scope and spirit of this disclosure. Accordingly, the following description should not be interpreted as limiting in any way.

The present disclosure relates to a storage device 100 configured to restrict unauthorized access, for example, by children and pets. This feature may be especially useful in cases where the contents include potentially hazardous materials, such as medications, chemical substances, sharp instruments, or other items requiring controlled and secure storage. In certain embodiments, the device may be further configured to provide theft deterrence through structural reinforcements or integrated locking mechanisms.

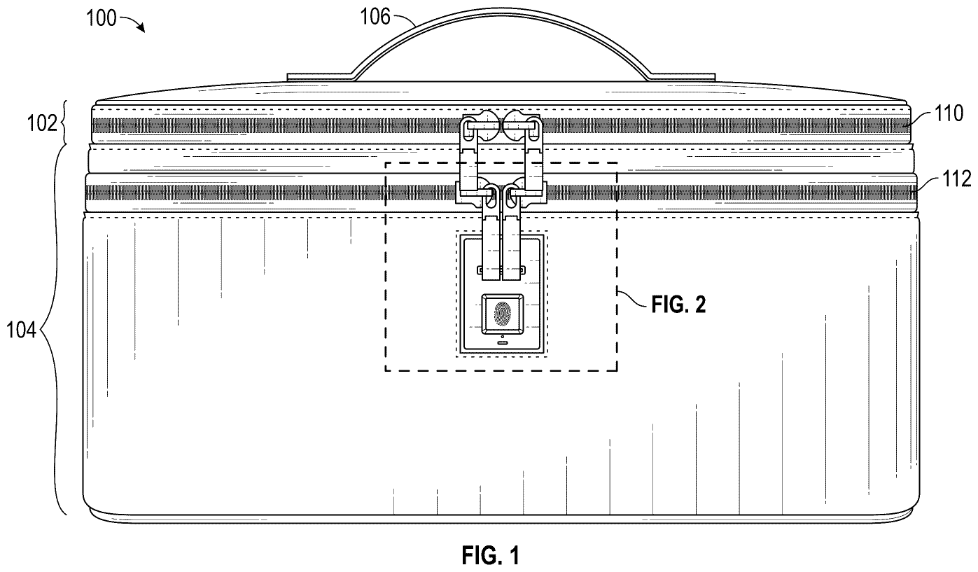

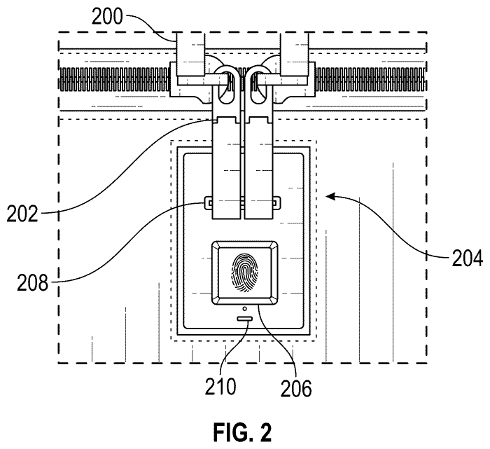

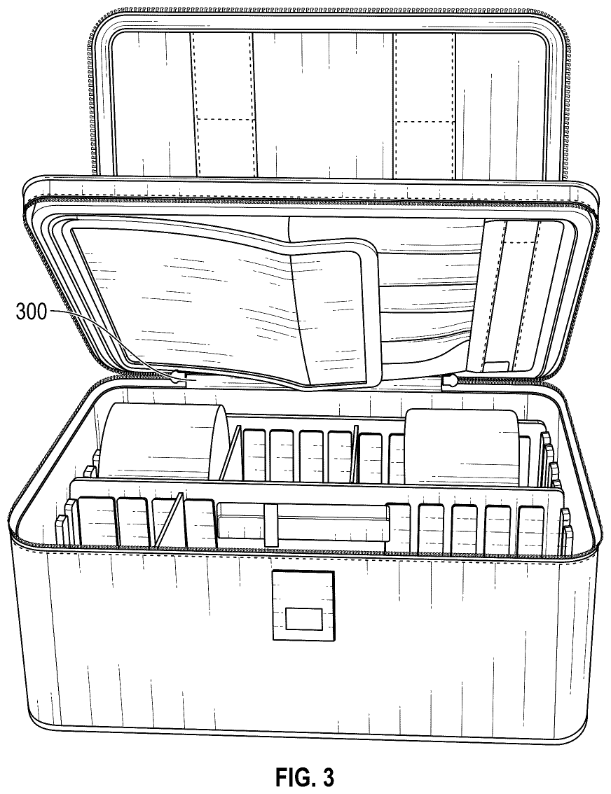

In one embodiment, the storage device 100 comprises a plurality of compartments configured to hold various products, packages, or accessories. As illustrated in FIG. 1, the device may include a first compartment 102 and a second compartment 104, though any suitable number of compartments may be used. A handle 106 may be attached to the top of the device to facilitate manual transport. The compartments 102, 104 may be operably connected via a hinge member 300, allowing them to pivot between open and closed configurations. The hinge member 301) is not visible in FIG. 1 but is shown in greater detail in FIG. 3. In certain embodiments, each compartment 102, 104 is enclosed by a respective fastening mechanism 110, 112, which in the illustrated embodiment comprises a zipper closure. For ease of reference, the fastening mechanism 110 on the upper (first) compartment 102 may be referred to as the upper zipper, while the fastening mechanism 112 on the lower (second) compartment may be referred to as the lower zipper. The upper (first) compartment 102 is accessed via an upper zipper 110, while the lower compartment 104 is accessed via a lower zipper 112. These zipper closures may include lockable tabs or be integrated with biometric authentication components to prevent unauthorized access. Each compartment is independently sealable. In certain embodiments, the compartments may only be opened upon manipulation of an integrated locking mechanism-such as a biometric sensor or coded latch—as further shown in FIG. 2. These features collectively contribute to both safety and portability.

FIG. 2 illustrates a close-up view of the locking mechanism located at the intersection of the upper (first) compartment 102 and lower (second) compartment 104, as identified in FIG. 1. The illustration highlights a pair of zipper tabs 200, 202, each corresponding to a respective compartment, that are configured to interlock at a central locking assembly 204. The locking assembly 204 includes a biometric authentication module 206, such as a fingerprint sensor, that is operable to selectively release the interlocked zipper tabs upon successful identification. In certain embodiments, each zipper tab 200, 202 includes a projecting member or anchor 208 that can be inserted into a corresponding receiving cavity or slot within the locking assembly 204, such that the tabs are held in a locked configuration until biometric access is granted. In certain embodiments, the locking assembly 204 may further include an auxiliary access port 210, such as the slot positioned below the fingerprint sensor, which may optionally accommodate a mechanical key, a digital code input, or an emergency override tool.

As illustrated in FIG. 1 and FIG. 2, each customizable compartment 102, 104 may include a fastening mechanism 110, 112 configured to fasten a perimeter of each customizable compartment to one or more adjacent customizable compartments 102, 104. As such, a user may selectively access one or more of the customizable compartments 102, 104 by interacting with one or more of the fastening mechanisms 110, 112. The fastening mechanism 110, 112 may include a zipper, as shown in the figures disclosed herein. However, other fastening mechanisms 110, 112 may be implemented as desired. For example, the fastening mechanism 110, 112 may include hook and loop, magnets, slider seal, or other suitable mechanism as may be known by those of skill in the art. The fastening mechanism 110, 112 may be secured via a locking mechanism 204, which may prevent any of the one or more customizable compartments 102, 104 from being accessed. The locking mechanism 204 is discussed in further detail below.

As previously discussed, FIG. 2 illustrates a locking mechanism 204 disposed at the convergence point of the zipper tabs 200, 202 corresponding to the upper and lower compartments. The locking mechanism 204 is configured to secure the fastening mechanisms 110, 112, thereby preventing any of the one or more customizable compartments 102, 104 from being accessed by an unauthorized user, such as a child.

In embodiments where the fastening mechanisms 110, 112 are zippers, the locking mechanism 204 may include a pin latch or similar device. For example, the locking mechanism 204 may comprise a sliding or rotating pin configured to pass through one or more holes defined within the zipper tabs 200, 202, thereby physically locking the tabs in place and preventing movement along the zipper track.

The locking mechanism 204 may be actuated electronically via an authentication module, such as a fingerprint sensor 206. The locking mechanism 204 may include a processor and computer-readable memory configured to store one or more authorized fingerprints. Upon user contact with the fingerprint sensor 206, the processor compares the scanned fingerprint against stored records. If a match is detected, the locking mechanism 204 disengages the latch and allows the zipper tabs to move freely; otherwise, the mechanism remains in a locked state.

In some embodiments, the locking mechanism 204 may be powered by a power supply, such as a rechargeable battery or replaceable cell, housed within or adjacent to the locking mechanism 204. The power supply may be electrically connected to the locking mechanism via internal wiring, and may be positioned anywhere along the body of the storage device 100 in a concealed or protected location.

While the power supply may be dedicated solely to the locking mechanism 204, it is contemplated that it may also be used to provide power to other electronic features, such as charging ports for external devices stored within the storage device 100, including but not limited to mobile phones or wearable accessories.

The customizable compartments 102, 104 may be independently customized to a desired depth and to include one or more customizable features as disclosed herein. FIG. 3 illustrates an exemplary configuration of the first compartment 102, including several of these features. FIG. 3 also illustrates an example of the customizable depth of the one or more compartments 102, 104. In the embodiment shown, the second bottom surface 504 is configured with a depth suitable for storing one or more larger items. The depth of each compartment may vary depending on the size of the items to be stored, and may, in some embodiments, fall within the range of approximately 3 to 12 inches. Other depth ranges are also contemplated.

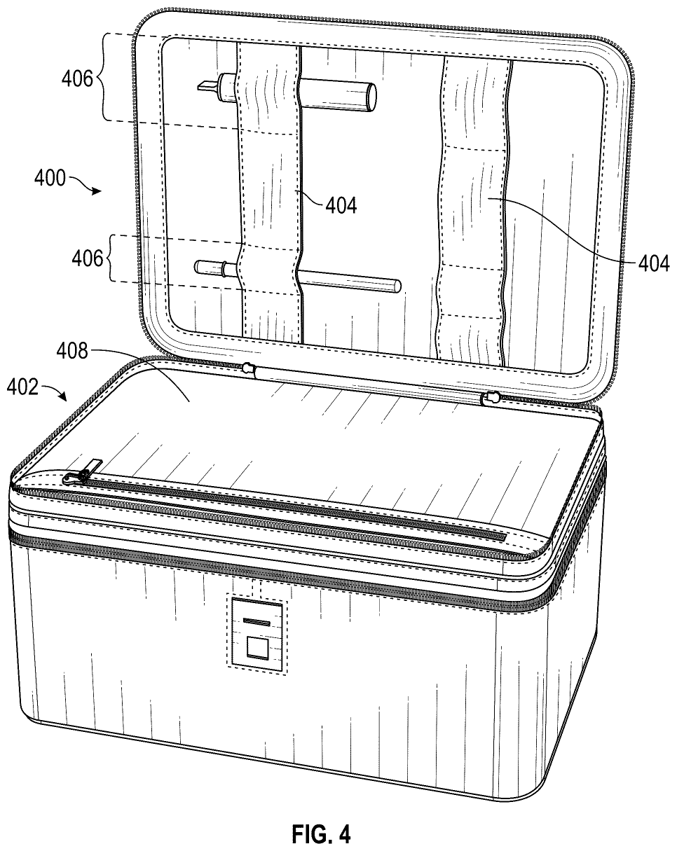

As exemplified in FIG. 3 and FIG. 4, each compartment 102, 104 may include a top surface 400 and a bottom surface 402. In this embodiment, the top surface 400 may include one or more elastic securing bands 404. As a non-limiting example, the one or more elastic securing bands 404 may include an elastic material or a non-elastic material interwoven with elastic fibers. As such, the one or more elastic securing bands 404 may be used to securely hold items between the band 404 and the top surface 400.

In an embodiment, the one or more elastic securing bands 404 may be attached, via stitching or other suitable attachment means, to the top surface 400 at several locations. In this embodiment, such attachments may define one or more securing widths 406, which may be customized to allow for the securing of items of different sizes. For example, the one or more securing widths 406 may be configured to hold items of several widths as exemplified in FIG. 4.

While FIG. 4 illustrates permanent means for attaching the one or more elastic securing bands 404 to the top surface 400, non-permanent attachment means may be utilized, such as magnets or hook and loop fasteners, to allow a user to customize the one or more securing widths 406.

The bottom surface 402 may include at least one pouch 408. The at least one pouch 408 may include means for opening or closing the at least one pouch 408, such as a zipper or other suitable mechanism as described herein. In an embodiment, and as illustrated in FIG. 4, the bottom surface 402 may include one pouch 408. However, there may be any quantity of pouches 408 as desired.

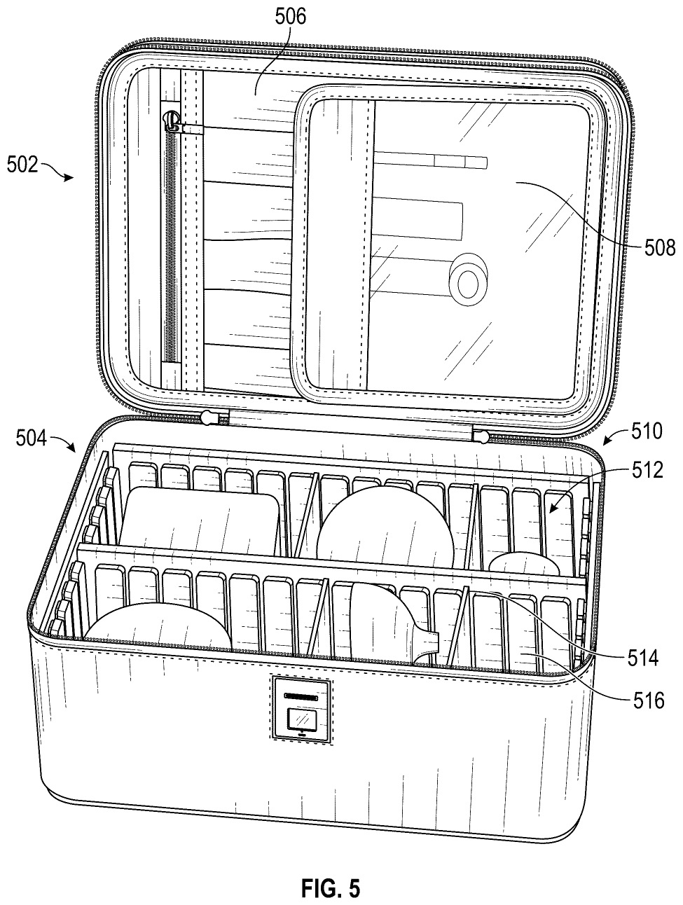

The storage device 100 may include a second compartment 104, as discussed above. An exemplary embodiment of the second compartment 104 is shown in the FIG. 5.

The second compartment 104 may include a second top surface 502 and a second bottom surface 504. Referring to the second top surface 502, it may incorporate a combination of organizational features, as shown in FIG. 5. Specifically, the second top surface 502 may include one or more open-ended pouches 506. In some embodiments, the one or more open-ended pouches 506 may be arranged side-by-side, as illustrated, or may alternatively be stacked vertically or arranged in multiple rows.

The one or more open-ended pouches 506 may each include a securing flap 508 configured to prevent contents from falling out. The securing flap 508 may be attached using non-permanent fastening means, such as hook-and-loop fasteners, magnetic attachments, or any other suitable non-permanent attachment method known to those of skill in the art.

In certain embodiments, the securing flap 508 may be composed of a transparent material, allowing the user to view the contents of the one or more open-ended pouches 506. In other embodiments, the flap 508 may be made of opaque or translucent materials to provide selective visibility, depending on the desired level of concealment.

The second bottom surface 504 may further include a divider system 510 configured to selectively define one or more storage sections 512. The divider system 510 may include one or more slots 514 and dividers 516. In one embodiment, the one or more slots 514 are disposed along the perimeter of the second bottom surface 504. In another embodiment, the one or more slots 514 may additionally or alternatively be disposed on the surface of one or more dividers 516, thereby allowing the dividers 516 to interconnect with each other and with the bottom surface 504 to form one or more storage sections 512. A user may vary the number and placement of dividers 516 to define a desired number of storage sections 512.

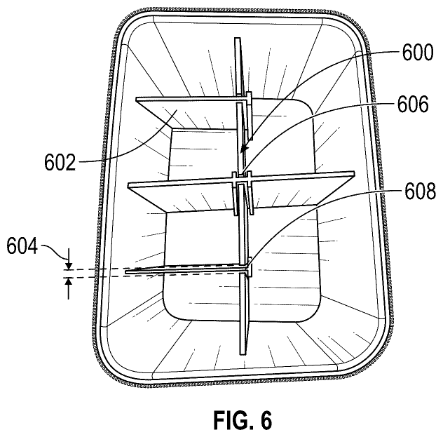

FIG. 6 illustrates an embodiment of a removable divider system 600 configured for insertion into one or more compartments of the storage device 100. The divider system 600 comprises one or more dividers 602, each configured to interconnect with at least one other divider 602 to define a modular grid of intersecting storage sections.

In the embodiment shown, the dividers 602 include one or more interlocking slots 604 disposed along the length of each divider. These slots 604 enable the dividers 602 to be inserted into one another in an orthogonal configuration, thereby forming a freestanding lattice structure that may be placed within a compartment. The dividers 602 may include rounded or angled ends to conform to the interior dimensions of the compartment. As further illustrated, the interlocking configuration of dividers 602 forms an engagement interface 606, enhancing structural stability within the divider system 600.

The removable divider system 600 may be inserted as a single unit or assembled in place within a compartment. In some embodiments, the dividers 602 may be composed of a semi-rigid material such as plastic, reinforced cardboard, or foam core. In alternative embodiments, the divider system 600 may further include attachment or stabilization features, such as magnetic bases, adhesive strips, hook and loop pads, or physical retention grooves disposed along the compartment walls.

The interlocking slot 604 may include a slot geometry 608 designed to facilitate frictional retention, such as a narrowed throat, beveled entry, or chamfered locking edge. In some embodiments, the engagement interface 606 may further include optional reinforcing features such as flexible snap ridges, notch grooves, or magnetic contact points.

Finally, other implementations of the disclosure will be apparent to those skilled in the art from consideration of the specification and practice of the disclosure disclosed herein. It is intended that the specification and examples be considered as exemplary only. The true scope and spirit of the disclosure are indicated by the following claims.

What is claimed is:

1. A storage device, comprising:
one or more compartments, each including:
a securing mechanism disposed along a perimeter of at least one compartment;
wherein the securing mechanism includes at least one zipper tab,
a divider system comprising one or more dividers and one or more corresponding slots, the divider system configured to define one or more customizable storage sections;
a locking mechanism comprising a latch and at least one authentication module;
wherein the authentication module comprising a fingerprint sensor or another form of user verification;
wherein the locking mechanism is configured to transition from the locked configuration to an unlocked configuration upon successful authentication.

2. The storage device of claim 1, wherein the locking mechanism comprises a receiving cavity configured to receive and retain at least a portion of each zipper tab in a locked configuration.

3. The storage device of claim 1, wherein the authentication module comprises at least one of:
biometric sensor, keypad, wireless reader, or other form of user verification system.

4. The storage device of claim 1,
wherein the locking mechanism further comprises at least one fallback unlocking mechanism,
wherein the fallback unlocking mechanism comprising a mechanical key interface, emergency override switch, or other non-biometric access structure.

5. The storage device of claim 1, wherein the divider system comprises a removable grid structure including two or more interlocking dividers, each divider including one or more interlocking slots configured to receive another divider in an orthogonal orientation.

6. The storage device of claim 5,
wherein the interlocking slots are formed along a length of each divider, and
wherein the slots enable the dividers to be assembled into a freestanding modular lattice.

7. The storage device of claim 1, wherein at least one compartment includes one or more elastic securing bands attached to an interior surface, the securing bands being configured to retain items between the band and the surface.

8. The storage device of claim 1, wherein at least one compartment includes one or more open-ended pouches, each pouch comprising a securing flap composed of a transparent, opaque, or translucent material, and configured to be removably fastened via a hook-and-loop fastener, magnet, or other non-permanent attachment mechanism.

9. The storage device of claim 1,
wherein the locking mechanism is powered by a power supply, and
wherein the power supply is further configured to charge or supply power to an external device stored within the storage device.

10. The storage device of claim 1, wherein one or more compartments are removably attachable to a body of the storage device, thereby allowing reconfiguration or replacement of the compartments.
